(19) United States
(12) Patent Application Publication
(10) Pub. No.: US 2019/0307315 A1
(43) Pub. Date: Oct. 10, 2019

(54) ENDOSCOPE SYSTEM

(71) Applicant: OLYMPUS CORPORATION, Tokyo (JP)

(72) Inventors: Yoshiro OKAZAKI, Tokyo (JP); Naoya SUGIMOTO, Tokyo (JP); Kazutoshi KUMAGAI, Tokyo (JP)

(73) Assignee: OLYMPUS CORPORATION, Tokyo (JP)

(21) Appl. No.: 16/417,801

(22) Filed: May 21, 2019

Related U.S. Application Data
(63) Continuation of application No. PCT/JP2016/085311, filed on Nov. 29, 2016.

(51) Int. Cl. A61B 1/00

(52) U.S. Cl. CPC A61B 1/00135

(57) ABSTRACT

An endoscope system is provided with: a sheath; an endoscope; a field-of-view ensuring part that has an expansion member formed of a sheet curved about the longitudinal axis of the sheath and a drive member generating an expansion force for expanding the expansion member in the radial direction and that is configured to project from and retract into the distal end of the sheath. The expansion member has a slit extending from the distal end toward the base end and is deformed from an expanded form in which expansion member has a larger outer diameter than the outer diameter of the sheath into a contracted form in which expansion member has a smaller outer diameter than the inner diameter of the sheath, by being rolled about the longitudinal axis.

### CROSS-REFERENCE TO RELATED APPLICATIONS

This is a continuation of International Application PCT/JP2016/085311, with an international filing date of Nov. 29, 2016, which is hereby incorporated by reference herein in its entirety.

### TECHNICAL FIELD

The present invention relates to an endoscope system, and in particular, to an endoscope system for the pericardial sac.

### BACKGROUND ART

In the related art, there is a known method in which an endoscope is inserted into a pericardial cavity from under the xiphoid, to observe a disease site without performing open-heart surgery (for example, see US Unexamined Patent Application Publication No. 2004/0064138). The pericardium, which covers the surface of the heart, is in close contact with the surface of the heart, and almost no gap exists between the surface of the heart and the pericardium. Therefore, in order to observe the surface of the heart through an endoscope inserted into a pericardial cavity, it is necessary to ensure a space between a distal end of the endoscope and the surface of the heart. However, since a pressing force toward the heart is applied from the pericardium to the endoscope when inserted into the pericardial cavity, it is difficult to freely operate the endoscope in the pericardial cavity and to maintain the distal end of the endoscope at a position away from the surface of the heart.

On the other hand, a device that ensures a space for observation using an endoscope in a digestive tract has been proposed (for example, see Japanese Unexamined Patent Application, Publication No. 2013-183895). The device described in Japanese Unexamined Patent Application, Publication No. 2013-183895 is made of a material having such elasticity as to be expandable and contractible between a cylindrical shape and a trumpet shape. This device is projected from the distal end of a sheath, into which the endoscope is inserted, and is expanded into the trumpet shape, thereby making it possible to ensure a sufficient field of view of the endoscope.

### SUMMARY OF INVENTION

According to one aspect, the present invention provides an endoscope system including: a sheath that is formed in a cylindrical shape and that has a longitudinal axis and that opens at both ends thereof; an endoscope that is inserted into the sheath; and a field-of-view ensuring part that is disposed in the sheath and that is configured to project from and retract into the distal end of the sheath, wherein the field-of-view ensuring part is provided with an expansion member that is formed of a sheet-like member curved about the longitudinal axis and formed into a substantially cylindrical shape and that is deformed between a contracted form in which the expansion member has a smaller outer diameter than an inner diameter of the sheath and an expanded form in which the expansion member has a larger outer diameter than an outer diameter of the sheath, and a drive member that is configured to generate an expansion force for deforming the expansion member from the contracted form into the expanded form; and the expansion member has a slit extending from a distal end toward a base end, at least at one place in the circumferential direction and is deformed from the expanded form into the contracted form by being rolled about the longitudinal axis such that at least ends that are adjacent to the slit are overlapped in the radial direction.

In the above-described aspect, the expansion member may have, in the expanded form, a shape extending over more than half the circumference about the longitudinal axis.

In the above-described aspect, the expansion member may have, in the expanded form, a shape whose diameter gradually increases from the base end toward the distal end.

In the above-described aspect, the expansion member may have an opening section that opens in the longitudinal direction of the expansion member in the expanded form.

In the above-described aspect, the field-of-view ensuring part may have a plurality of the expansion members arranged in the circumferential direction around the longitudinal axis; and ends of two of the expansion members adjacent in the circumferential direction may be overlapped at least at base end sections thereof.

### BRIEF DESCRIPTION OF DRAWINGS

FIG. 1A is a view showing a state in which a field-of-view ensuring part is stored in a sheath, in an endoscope system according to a first embodiment of the present invention.

FIG. 1B is a view showing a state in which the field-of-view ensuring part is projected to the outside of the sheath, in the endoscope system according to the first embodiment of the present invention.

FIG. 2 is a view for explaining the operation of the endoscope system, shown in FIGS. 1A and 1B, in a pericardial cavity.

FIG. 3 is a view showing a modification of an expansion member in the endoscope system shown in FIGS. 1A and 1B.

FIG. 4 is a view showing another modification of the expansion member in the endoscope system shown in FIGS. 1A and 1B.

FIG. 5A is a view showing a state in which a field-of-view ensuring part is stored in the sheath, according to a modification of the endoscope system shown in FIGS. 1A and 1B.

FIG. 5B is a view showing a state in which the field-of-view ensuring part is projected to the outside of the sheath, according to the modification of the endoscope system shown in FIGS. 1A and 1B.

FIG. 6A is a view showing a state in which a field-of-view ensuring part is stored in the sheath, in an endoscope system according to a second embodiment of the present invention.

FIG. 6B is a view showing a state in which the field-of-view ensuring part is projected to the outside of the sheath, in the endoscope system according to the second embodiment of the present invention.

FIG. 7A is a view showing a state in which a field-of-view ensuring part is stored in the sheath, in an endoscope system according to a third embodiment of the present invention.

FIG. 7B is a view showing a state in which the field-of-view ensuring part is projected to the outside of the sheath, in the endoscope system according to the third embodiment of the present invention.

### DESCRIPTION OF EMBODIMENTS

### First Embodiment

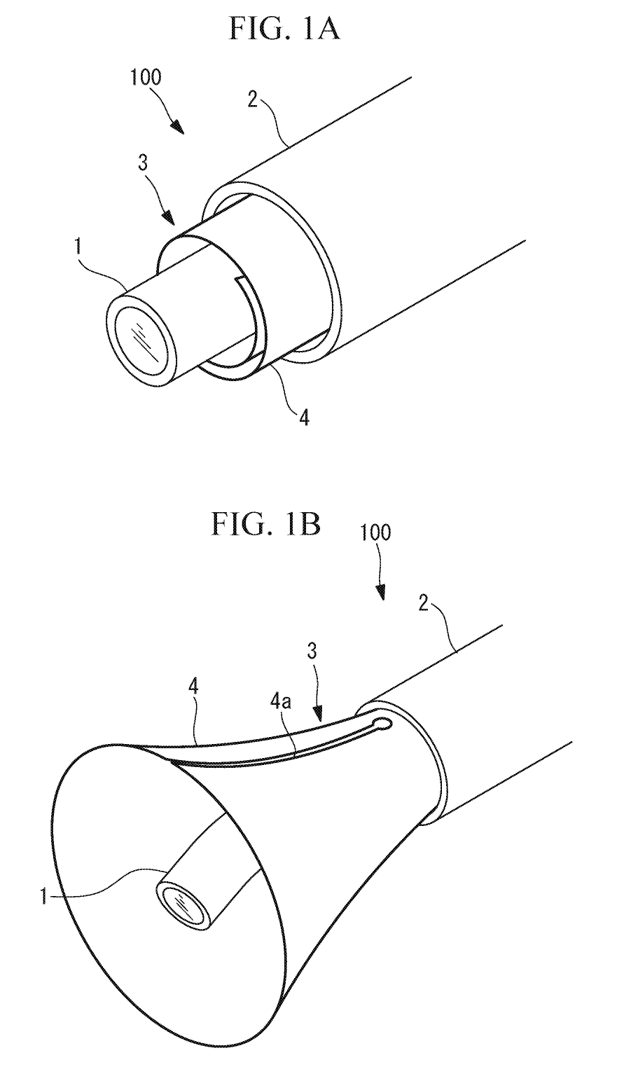

An endoscope system 100 according to a first embodiment of the present invention will be described below with reference to FIGS. 1A to 5B.

As shown in FIGS. 1A and 1B, the endoscope system 100 of this embodiment is provided with: an endoscope 1; a sheath 2 formed in a cylindrical shape and into which the endoscope 1 is inserted; and a field-of-view ensuring part 3 that is disposed around a distal end section of the endoscope 1 and that is projectable from and retractable into a distal end of the sheath 2. FIG. 1A shows a state in which the field-of-view ensuring part 3 is stored in the sheath 2, and FIG. 1B shows a state in which the field-of-view ensuring part 3 is projected to the outside of the sheath 2.

The endoscope 1 is a thin flexible endoscope used for the pericardial sac.

The sheath 2 has such flexibility as to be bendable according to the shape of tissue in the body. The sheath 2 opens at both ends such that the endoscope 1 can be inserted thereinto so as to penetrate therethrough, and the sheath 2 and the endoscope 1 disposed in the sheath 2 are movable relative to each other in the longitudinal direction. As shown in FIG. 1A, when the distal end of the sheath 2 is disposed near a distal end of the endoscope 1, the field-of-view ensuring part 3 is stored in the sheath 2, and, as shown in FIG. 1B, by moving the sheath 2 toward the base end with respect to the endoscope 1, the field-of-view ensuring part 3 is projected from the distal end of the sheath 2.

The field-of-view ensuring part 3 is provided with an expansion member (drive member) 4 that is formed of a single continuous resin sheet having elasticity, that is bent about the longitudinal axis of the sheath 2, and that is formed into a tube shape. The material of the resin sheet is, for example, a polyurethane-based shape memory polymer or a thermosetting resin. As shown in FIG. 1B, in a natural state in which an external force does not act, the expansion member 4 has an expanded form whose diameter gradually increases from the base end toward the distal end and that has a substantially circular-truncated-cone cylindrical shape over the entire circumference. In the expanded form, the distal end of the expansion member 4 has a larger outer diameter than the outer diameter of the sheath 2. The expansion member 4 has such rigidity as to be able to keep the expanded form against a pressing force in the radial direction received from the pericardium in the pericardial cavity.

The expansion member 4 has, at one place in the circumferential direction, a slit 4a that is formed from the distal end toward the base end in a generatrix direction and that divides the expansion member 4 in the circumferential direction. As shown in FIG. 1A, the expansion member 4 is elastically deformed into a contracted form having a substantially cylindrical shape whose outer diameter is smaller than the inner diameter of the sheath 2, by being rolled around the side surface of the endoscope 1 such that at least both ends of the expansion member 4 in the circumferential direction are overlapped in the radial direction.

In the contracted form, the expansion member 4 is disposed in a cylindrical space between the side surface of the endoscope 1 and an inner surface of the sheath 2 so as to cover the distal end section of the endoscope 1. When the sheath 2 is pulled toward the base end, the expansion member 4 is projected to the outside of the sheath 2 while being gradually deformed into the expanded form by being expanded gradually from the distal end. When the sheath 2 is pushed toward the distal end, the expansion member 4 is stored in the sheath 2 while being gradually deformed into the contracted form by being rolled gradually from the base end. In order to facilitate deformation of the expansion member 4 into the contracted form in the process of storage in the sheath 2, it is preferred that the expansion member 4 in the expanded form be formed such that, at the slit 4a, one end of the expansion member 4 in the circumferential direction is positioned closer to a radially inner side than the other end thereof is.

Next, the operation of the thus-configured endoscope system 100 will be described.

In order to observe the heart by using the endoscope system 100 of this embodiment, the sheath 2 is inserted into the body from under the xiphoid together with the endoscope 1 and the field-of-view ensuring part 3, which are stored in the sheath 2, and the distal end section of the sheath 2 is disposed in a pericardial cavity. Next, by pulling the sheath 2 toward the base end while maintaining the positions of the endoscope 1 and the field-of-view ensuring part 3, the field-of-view ensuring part 3 is projected from the distal end of the sheath 2.

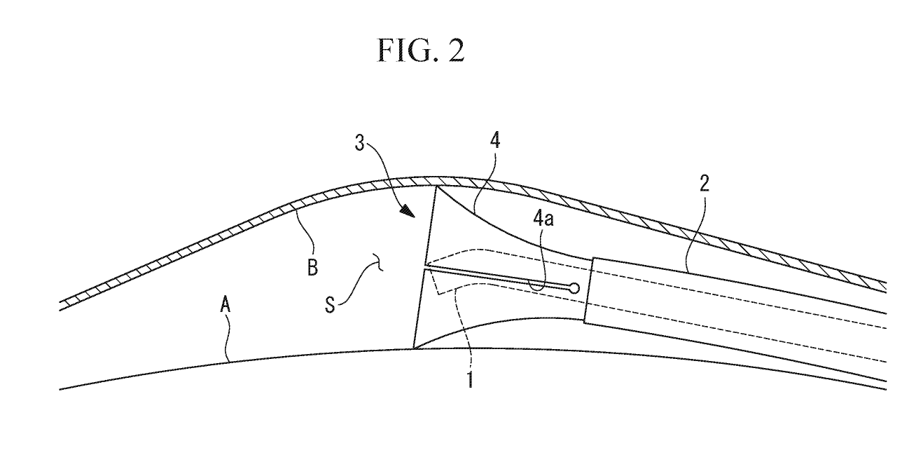

As shown in FIG. 2, the expansion member 4 disposed outside the sheath 2 self-expands into the expanded form while pressing a pericardium B upward by using an elastic restoring force of the expansion member 4 as an expansion force. Because the expansion member 4 has such rigidity as to resist the pressing force from the pericardium B toward the heart A, it is possible to maintain the pericardium B at a position away from the surface of the heart A. Accordingly, a space S is ensured in front of the distal end of the sheath 2, thus making it possible to dispose the distal end of the endoscope 1 at a position away from the surface of the heart A and to enable overview observation of the surface of the heart A through the endoscope 1.

In this way, according to this embodiment, through a simple operation of merely pulling the sheath 2, it is possible to ensure the space S in front of the distal end of the sheath 2 and to observe the heart A by means of the endoscope 1. Because the expansion member 4, which is formed of a resin sheet having a substantially circular-truncated-cone cylindrical shape, is provided with the slit 4a extending in the generatrix direction, even if a material having relatively high rigidity is selected as the material of the expansion member 4, it is possible to easily elastically deform the expansion member 4 into the contracted form, in which the expansion member 4 can be stored in the sheath 2, thus achieving ease of use.

By changing the projection amount of the expansion member 4 projected from the distal end of the sheath 2, the diameter of the expansion member 4 at the distal end thereof changes. Therefore, by adjusting the pulling amount of the sheath 2, it is possible to adjust the size of the space S to be ensured by the expansion member 4.

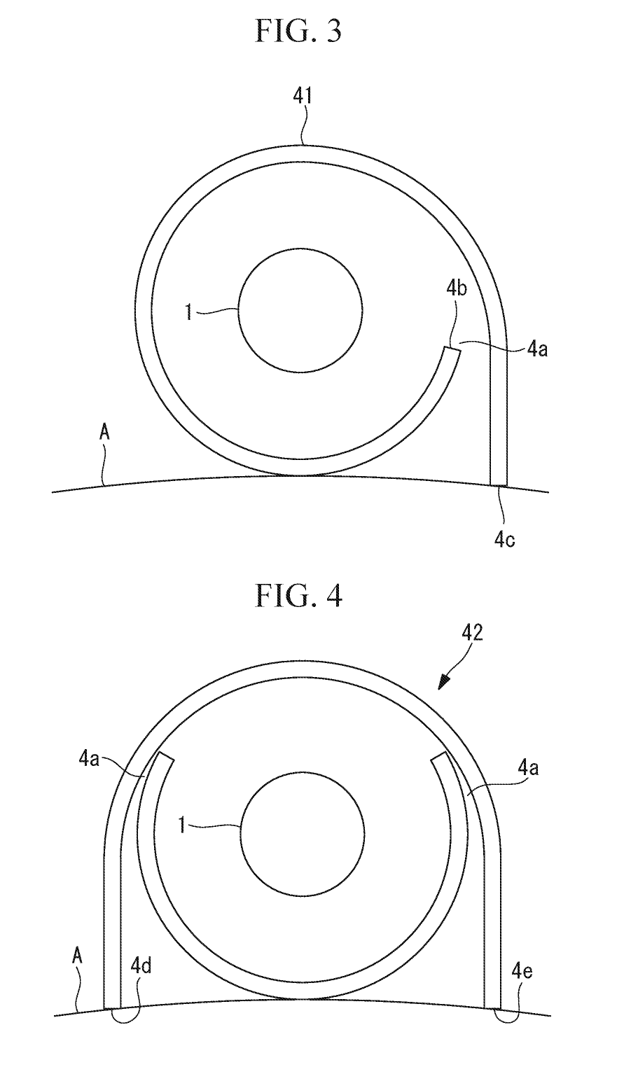

In this embodiment, as shown in FIG. 3, an expansion member 41 in the expanded form may have a shape rolled, over more than one turn, in a substantially circular-truncated-cone cylindrical manner such that both circumferential ends thereof are overlapped in the radial direction.

In this case, of two circumferential ends 4b and 4c of the expansion member 41, the end 4c, which is disposed at an outer side, is brought into contact with the surface of the heart A, thus supporting the expansion member 41 at the end 4c. Accordingly, the expanded form of the expansion member 41 can be stabilized even more.

In this embodiment, although the expansion member 4 is provided with only one slit 4a, instead of this, it is also possible to provide two or more slits 4a at intervals in the circumferential direction.

In this case, as shown in FIG. 4, because an expansion member 42 in the expanded form is supported at two ends 4d and 4e along the longitudinal direction, the expanded form of the expansion member 42 can be stabilized even more. The expansion member 42 is brought into contact with the surface of the heart A at the ends 4d and 4e of the slits 4a, thus making it possible to position the expansion member 42 and the endoscope 1 with respect to the heart A.

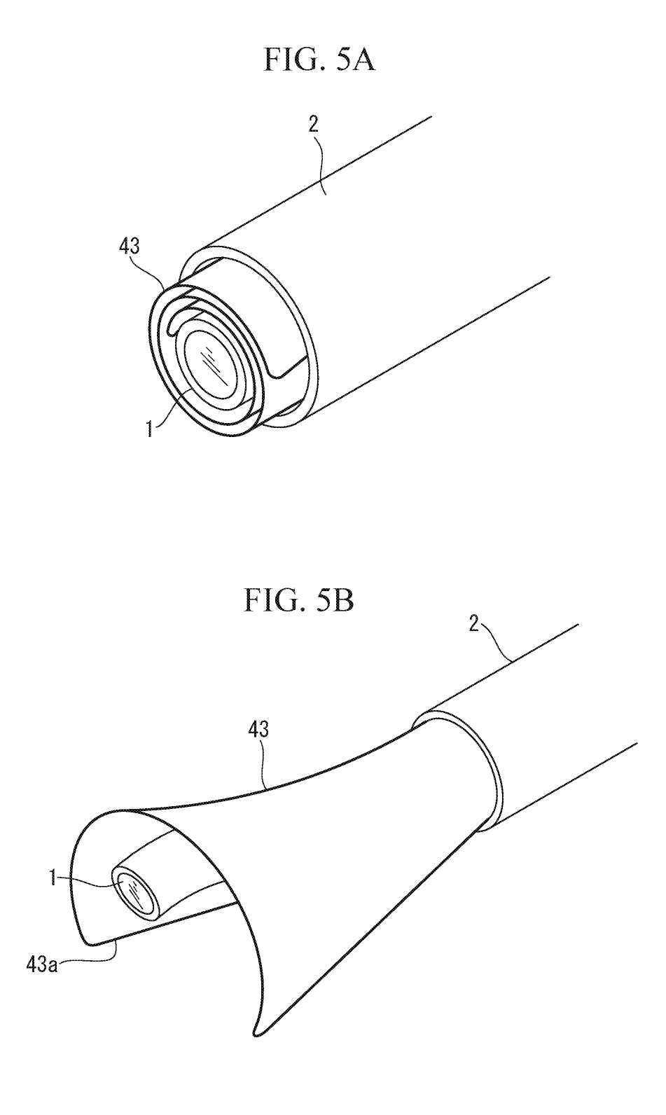

In this embodiment, although the expansion member 4 in the expanded form has a substantially circular-truncated-cone cylindrical shape over the entire circumference, instead of this, as shown in FIGS. 5A and 5B, it may have a substantially partial circular-truncated-cone cylindrical shape in which a circumferential section of the cone is notched in the longitudinal direction. In this case, it is preferred that an expansion member 43 in the expanded form have a shape extending over more than half the circumference in the circumferential direction.

In this modification, a transverse cross section of the expansion member 43 in the expanded form, in the radial direction, is substantially U-shaped or substantially C-shaped, and an opening section 43a that is formed of a large width slit and that communicates the inside and the outside of the expansion member 43 with each other is formed at one radial side of the expansion member 43 in the expanded form. Therefore, as shown in FIG. 5B, the expansion member 43 is disposed such that the circumferential ends of the expansion member 43 are both brought into contact with the surface of the heart A, thereby supporting the expansion member 43 at two places, thus making it possible to more stably maintain the expanded form with respect to the pressing force from the pericardium in the radial direction. Because the surface of the heart A is exposed to the inside of the expansion member 43 via the opening section 43a, it is possible to observe a wider region of the surface of the heart A through the endoscope 1.

In this modification, as shown in FIG. 5B, the endoscope 1 may also be disposed at an eccentric position with respect to the expansion member 43 in the expanded form, more preferably at an eccentric position toward the opposite side from the opening section 43a. By doing so, because a larger space is ensured on the side of the endoscope 1 close to the opening section 43a, it is possible to observe the surface of the heart A by bending, inside the expansion member 43, a bending section of the endoscope 1 toward the opening section 43a. The expansion member 43 is brought into contact with the surface of the heart A at both ends of the slit (opening section 43a), thus making it possible to position the expansion member 43 and the endoscope 1 with respect to the heart A.

In this embodiment, it is also possible to provide an opening section in the expansion member 4 by making a hole in a part of the expansion member 4.

### Second Embodiment

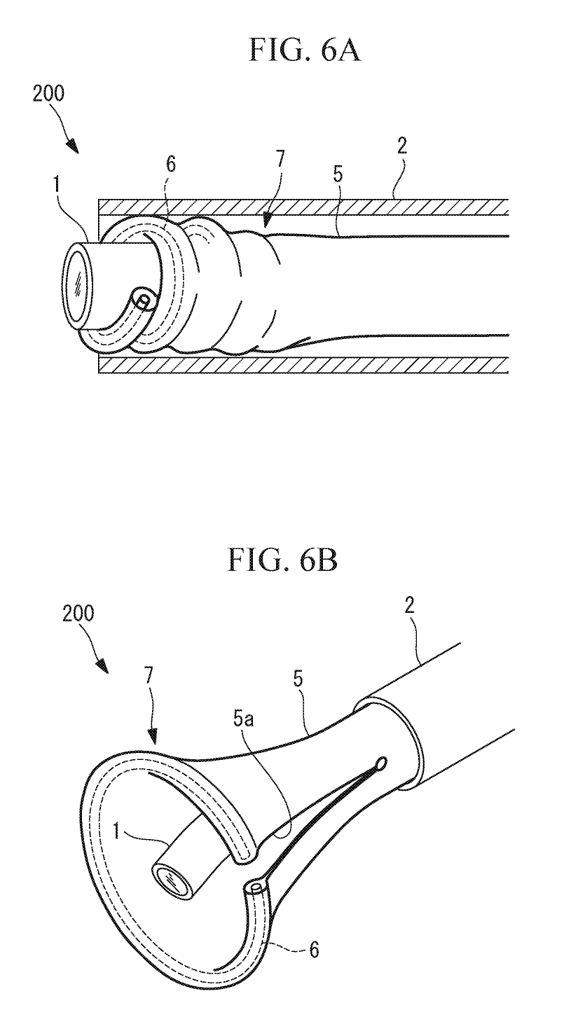

Next, an endoscope system according to a second embodiment of the present invention will be described with reference to FIGS. 6A and 6B. In this embodiment, structures that are different from those in the first embodiment will be mainly described, identical reference signs are assigned to structures that are common to those in the first embodiment, and a description thereof will be omitted.

As shown in FIG. 6A, an endoscope system 200 of this embodiment is provided with: the endoscope 1; the sheath 2; and a field-of-view ensuring part 7 that has an expansion member 5 and a drive member 6.

The expansion member 5 is formed in the same way as the expansion member 4 of the first embodiment. However, the material of a resin sheet that forms the expansion member 5 need not be a shape memory polymer or a thermosetting resin and may be an elastic material that has low rigidity or a flexible material that does not have elasticity, such as ePTFE (expanded porous polytetrafluoroethylene) or FEP (tetrafluoroethylene-hexafluoropropylene copolymer resin).

The drive member 6 is formed of a wire that is made of a shape memory alloy, such as NiTi (nickel titanium) or βTi (beta titanium), and that memorizes a ring shape. The drive member 6 is provided at a distal end section of the expansion member 5 along the circumferential direction and is cut at one place in the circumferential direction corresponding to a slit 5a of the expansion member 5. In the example shown in FIGS. 6A and 6B, the drive member 6 is embedded in the distal end section of the expansion member 5, and circumferential ends of the drive member 6 are not exposed.

As shown in FIG. 6B, in a natural state in which an external force does not act, the drive member 6 has an expanded form that has a substantially torus shape whose outer diameter is larger than the outer diameter of the sheath 2. The drive member 6 has such rigidity as to be able to maintain the expanded form against a pressing force in the radial direction received from the pericardium in the pericardial cavity. As shown in FIG. 6A, the drive member 6 is elastically deformed into a contracted form that has a substantially spiral shape whose outer diameter is smaller than the inner diameter of the sheath 2, by being rolled such that at least both circumferential ends of the drive member 6 are overlapped in the radial direction. In order to further facilitate deformation of the drive member 6 into the contracted form, it is preferred that the drive member 6 in the expanded form be formed such that, at the slit 5a, one circumferential end of the drive member 6 is positioned closer to a radially inner side than the other end thereof is.

When the sheath 2 is pulled toward the base end, the drive member 6, which is provided at the distal end section of the expansion member 5, is disposed outside the sheath 2, thus being deformed into the expanded form due to an elastic restoring force of the drive member 6 itself. By using the elastic restoring force of the drive member 6 at this time, as an expansion force, the expansion member 5, which is closer to the base end than the drive member 6 is, is expanded into a substantially circular-truncated-cone cylindrical shape. When the sheath 2 is pushed toward the distal end, the expansion member 5 is stored in the sheath 2 while being gradually deformed into the contracted form by being rolled gradually from the base end. At this time, in accordance with the deformation of the expansion member 5 into the contracted form, the drive member 6 is also rolled in a spiral manner and is deformed into the contracted form.

Since the operation of this embodiment is the same as the first embodiment, a description thereof will be omitted.

According to this embodiment, in addition to the advantageous effect of the first embodiment, the following advantageous effect is afforded. Because the drive member 6, which is made of a shape memory alloy, generates a larger expansion force than the expansion member 4, which is formed of a resin sheet, in the first embodiment, shape reproducibility when the expansion member 5 is expanded can be improved. Because the elastic restoring force of the shape memory alloy is less subject to the influence of temperature compared with that of the resin sheet, a more stable expansion force can be exerted on the body.

### Third Embodiment

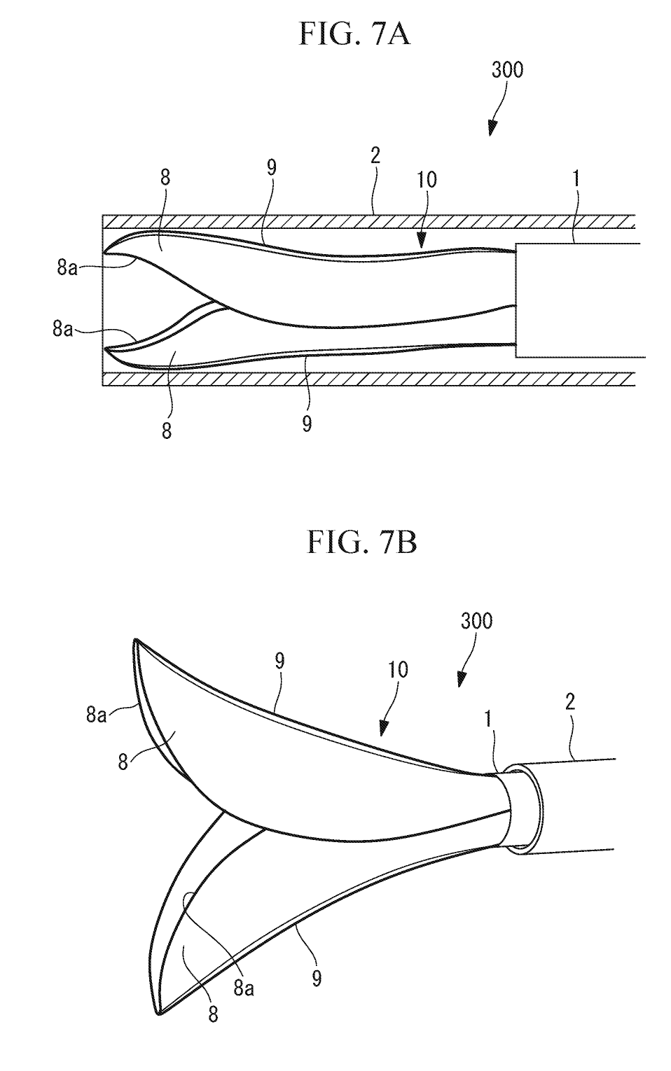

Next, an endoscope system 300 according to a third embodiment of the present invention will be described with reference to FIGS. 7A and 7B. In this embodiment, structures that are different from those in the first and second embodiments will be mainly described, identical reference signs are assigned to structures that are common to those in the first and second embodiments, and a description thereof will be omitted.

As shown in FIG. 7A, the endoscope system 300 of this embodiment is provided with: the endoscope 1; the sheath 2; and a field-of-view ensuring part 10 that has a plurality of pairs of expansion members 8 and drive members 9 and that is disposed at the distal end of the endoscope 1. Although FIGS. 7A and 7B show, as an example case, two pairs of expansion members 8 and drive members 9, three or more pairs of expansion members 8 and drive members 9 may be provided.

Each of the expansion members 8 is formed of a single continuous resin sheet having a shape that has a long axis and that is formed of a smooth curve, such as the shape of an ellipse, an oval, or an egg. The resin sheet may have elasticity, as in the resin sheet of the expansion member 5, but it need not have elasticity. Each of the expansion members 8 is fixed, at the base end thereof, to the distal end section of the endoscope 1 such that the long axis projects substantially along the longitudinal direction from the distal end of the endoscope 1, and is curved in the circumferential direction of the endoscope 1.

A plurality of expansion members 8, preferably, two expansion members 8, are arranged in the circumferential direction of the endoscope 1 so as to be opposed to each other in the radial direction of the endoscope 1. Therefore, in this embodiment, slits are provided between the two expansion members 8 adjacent in the circumferential direction. Circumferential ends of the two expansion members 8, which are adjacent in the circumferential direction, are overlapped at least at the base end sections thereof. Accordingly, an area near the distal end of the endoscope 1 is covered with the expansion members 8 over the entire circumference.

The drive members 9 are each formed of a wire that is made of a shape memory alloy, such as NiTi (nickel titanium) or βTi (beta titanium), and that memorizes a curve shape or a linear shape. The drive member 9 is provided on each of the expansion members 8 along the long axis thereof.

As shown in FIG. 7B, in a natural state in which an external force does not act, the drive member 9 has an expanded form in which the drive member 9 is inclined with respect to the longitudinal direction of the endoscope 1 so as to be gradually displaced outward in the radial direction from the base end toward the distal end. Therefore, the plurality of expansion members 8 take the expanded form having a substantially circular-truncated-cone cylindrical shape whose diameter gradually increases from the base end toward the distal end. The drive members 9 have such rigidity as to be able to maintain the expanded form against a pressing force in the radial direction received from the pericardium in the pericardial cavity. As shown in FIG. 7A, the drive members 9 are elastically deformed into the contracted form, in which the drive members 9 extend substantially linearly along the longitudinal direction of the endoscope 1. Therefore, the plurality of expansion members 8 can take the contracted form having a substantially cylindrical shape extending substantially in parallel from the base end to the distal end.

When the sheath 2 is pulled toward the base end, the drive members 9 are projected to the outside of the sheath 2 while being gradually deformed into the expanded form by being inclined outward in the radial direction gradually from the distal ends due to the elastic restoring forces thereof. By using the elastic restoring forces of the drive members 9 at this time, as expansion forces, the plurality of expansion members 8 are also gradually deformed into the expanded form by being expanded in the radial direction gradually from the distal ends. Here, the diameter of each of the expansion members 8 does not keep expanding from the base end to the distal end but contracts from a middle section toward the distal end, thus forming opening sections 8a along the longitudinal direction, between the two expansion members 8, which are adjacent in the circumferential direction.

When the sheath 2 is pushed toward the distal end, the plurality of drive members 9 are stored in the sheath 2 while being gradually deformed into the contracted form by being narrowed gradually from the base ends. In accordance with the deformation of the drive members 9, the plurality of expansion members 8 are also deformed into the contracted form by being narrowed gradually from the base ends. Note that, in FIG. 7B, at least one of the opening sections 8a, which are formed between the expansion members 8, is preferably disposed so as to be brought into contact with an observation target (for example, the heart A), as in FIG. 5B, thereby making it possible not only to achieve shape stabilization but also to obtain the field of view with respect to the observation target in the longitudinal direction toward the base end.

Since the operation of this embodiment is the same as the first embodiment, a description thereof will be omitted.

According to this embodiment, in addition to the advantageous effects of the first and second embodiments, the following advantageous effect is afforded. The field-of-view ensuring part 10 is composed of the plurality of expansion members 8, thereby making it possible to realize deformation between the contracted form and the expanded form by means of the drive members 9, which have simpler structures. The degree of freedom in design, such as the shapes of the expansion members 8, the sizes thereof, and the angles of inclinations thereof in the expanded form, is improved.

In the above-described first to third embodiments, although the field-of-view ensuring part 3, 7, 10 is projected from and retracted into the distal end of the sheath 2 by pushing or pulling the sheath 2, instead of this or in addition to this, the field-of-view ensuring part 3, 7, 10 may also be projected from and retracted into the distal end of the sheath 2 by moving the field-of-view ensuring part 3, 7, 10 with respect to the sheath 2 in the longitudinal direction. For example, it is also possible to provide an operating member (not shown) that is connected to the base end of the expansion member 4, 5, 8 and that extends to the outside from the base end of the sheath 2, and to move the field-of-view ensuring part 3, 7, 10 in the longitudinal direction by pushing or pulling the operating member.

As another usage of the endoscope system 100, 200, 300, there is a method in which only the sheath 2 is first inserted to a desired site in the pericardial cavity while observing the sheath 2 in the body by using a modality (for example, an ultrasound imaging device or an X-ray fluoroscopy device) other than the endoscope 1, and then, the endoscope 1 is inserted into the pericardial cavity. In such a case, the field-of-view ensuring part 3, 7, 10 is inserted into the sheath 2 at the same time as the endoscope 1 or after the endoscope 1 and is projected from the distal end of the sheath 2, which is positioned in the body, thereby making it possible to ensure a space in front of the distal end of the sheath 2.

The present invention is not limited to the above-described embodiments and can be variously modified without departing from the scope of the present invention. For example, in the expansion members 4, 5, shown in FIGS. 1B and 6B, the opening sections, which are formed of the slits 4a, 5a, are provided (or, the expansion members 4, 5 are manually rotated about the axis and are disposed) at a position to be brought into contact with an observation target (for example, the heart A), as shown in FIG. 5B, thus making it possible not only to achieve shape stabilization of the expansion members 4, 5 but also to obtain the field of view with respect to the observation target in the longitudinal direction toward the base end.

As a result, the following aspects are derived from the above-described embodiments.

According to one aspect, the present invention provides an endoscope system including: a sheath that is formed in a cylindrical shape and that has a longitudinal axis and that opens at both ends thereof; an endoscope that is inserted into the sheath; and a field-of-view ensuring part that is disposed in the sheath and that is configured to project from and retract into the distal end of the sheath, wherein the field-of-view ensuring part is provided with an expansion member that is formed of a sheet-like member curved about the longitudinal axis and formed into a substantially cylindrical shape and that is deformed between a contracted form in which the expansion member has a smaller outer diameter than an inner diameter of the sheath and an expanded form in which the expansion member has a larger outer diameter than an outer diameter of the sheath, and a drive member that is configured to generate an expansion force for deforming the expansion member from the contracted form into the expanded form; and the expansion member has a slit extending from a distal end toward a base end, at least at one place in the circumferential direction and is deformed from the expanded form into the contracted form by being rolled about the longitudinal axis such that at least ends that are adjacent to the slit are overlapped in the radial direction.

According to the present invention, the sheath is percutaneously disposed in a pericardial cavity from the outside of the body, the field-of-view ensuring part, which is disposed in the sheath and which includes the expansion member in the contracted form, is projected from the distal end of the sheath, and the expansion member is deformed into the expanded form, in which the expansion member has a larger diameter than the outer diameter of the sheath, by means of the drive member. The expansion member in the expanded form is disposed so as to cover an area in front of the distal end of the sheath, in the circumferential direction about the longitudinal axis of the sheath, and, in front of the distal end of the sheath, the pericardium is maintained at a position away from the surface of the heart by the expansion member. Accordingly, because a space is ensured in front of the distal end of the sheath, the surface of the heart can be observed through the endoscope, which is inserted into the pericardial cavity via the sheath.

In this case, the slit is formed in the expansion member, and the expansion member is deformed into the contracted form, in which the expansion member has a smaller outer diameter than the inner diameter of the sheath, by being rolled about the longitudinal axis. Therefore, even if the rigidity of the expansion member is increased such that the expansion member in the expanded form can withstand a force from the pericardium, the expansion member can be easily deformed from the expanded form into the contracted form. Accordingly, the expansion member can be restored from the expanded form to the contracted form and can be easily stored in the sheath, thus making it possible to achieve ease of use.

In the above-described aspect, the expansion member may have, in the expanded form, a shape extending over more than half the circumference about the longitudinal axis.

By doing so, a space can be more stably ensured in front of the distal end of the sheath.

In the above-described aspect, the expansion member may have, in the expanded form, a shape whose diameter gradually increases from the base end toward the distal end.

By doing so, it is possible to adjust the size of a space to be ensured in front of the distal end of the sheath, by changing the projection amount of the expansion member from the distal end of the sheath.

In the above-described aspect, the expansion member may have an opening section that opens in the longitudinal direction of the expansion member in the expanded form.

By doing so, the outside of the expansion member can be observed via the opening section through the endoscope. When the expansion member is disposed such that the opening section is positioned close to the heart, because the surface of the heart is exposed to the space ensured inside the expansion member, it is possible to observe the surface of the heart even inside the expansion member. The expansion member is disposed so as to be brought into contact with the surface of the heart at both ends of the opening section, thereby making it possible to stabilize the position of the expansion member and to position the expansion member with respect to the heart.

In the above-described aspect, the field-of-view ensuring part may have a plurality of the expansion members arranged in the circumferential direction around the longitudinal axis; and ends of two of the expansion members adjacent in the circumferential direction may be overlapped at least at base end sections thereof.

By doing so, compared with a case in which a single expansion member is provided, the degree of freedom in design of the expansion member can be improved. The expansion members are overlapped at base end sections thereof, thereby making it possible to smoothly deform the plurality of expansion members from the expanded form into the contracted form.

### REFERENCE SIGNS LIST

100, 200, 300 endoscope system

1 endoscope

2 sheath

3, 7, 10 field-of-view ensuring part

4, 41, 42, 43, 5, 8 expansion member

4a, 5a slit

43a opening section

6, 9 drive member

S space

A heart

B pericardium

1. An endoscope system comprising:
a sheath that is formed in a cylindrical shape and that has a longitudinal axis and that opens at both ends thereof;
an endoscope that is inserted into the sheath; and
a field-of-view ensuring part that is disposed in the sheath and that is configured to project from and retract into the distal end of the sheath,
wherein the field-of-view ensuring part is provided with
an expansion member that is formed of a sheet-like member curved about the longitudinal axis and formed into a substantially cylindrical shape and that is deformed between a contracted form in which the expansion member has a smaller outer diameter than an inner diameter of the sheath and an expanded form in which the expansion member has a larger outer diameter than an outer diameter of the sheath, and
a drive member that is configured to generate an expansion force for deforming the expansion member from the contracted form into the expanded form; and
the expansion member has a slit extending from a distal end toward a base end, at least at one place in the circumferential direction and is deformed from the expanded form into the contracted form by being rolled about the longitudinal axis such that at least ends that are adjacent to the slit are overlapped in the radial direction.

2. An endoscope system according to claim 1, wherein the expansion member has, in the expanded form, a shape extending over more than half the circumference about the longitudinal axis.

3. An endoscope system according to claim 1, wherein the expansion member has, in the expanded form, a shape whose diameter gradually increases from the base end toward the distal end.

4. An endoscope system according to claim 1, wherein the expansion member has an opening section that opens in the longitudinal direction of the expansion member in the expanded form.

5. An endoscope system according to claim 1,
wherein the field-of-view ensuring part has a plurality of the expansion members arranged in the circumferential direction around the longitudinal axis; and
ends of two of the expansion members adjacent in the circumferential direction are overlapped at least at base end sections thereof.
